SEQUENCE LISTING (9 sequences)

SEQ ID NO: 1 - DNA, 19 bp, Artificial
  hSPP2 consensus
cagaagggactttccttcc

SEQ ID NO: 2 - DNA, 20 bp, Artificial
  hSPP2 consensus
ctggtggggttttcccagtg

SEQ ID NO: 3 - DNA, 20 bp, Artificial
  NFkappaB consensus
cagagggggatttccaagag

SEQ ID NO: 4 - DNA, 26 bp, Artificial
  Sp1 consensus
ggaggcggggcaggggtgtggactcg

SEQ ID NO: 5 - DNA, 26 bp, Artificial
  Primer
caccatgtcgctgaggcagcgcctgg

SEQ ID NO: 6 - DNA, 33 bp, Artificial
  Primer
tcaagagataccaataaagaaaaatatgtaagg

SEQ ID NO: 7 - DNA, 64 bp, Artificial
  Primer
caccatggccgagctgctgcggagcctgcaggattcccagctcgtcgcccgcttccagcgccgc

SEQ ID NO: 8 - DNA, 32 bp, Artificial
  Primer
tcagggtaatcccagaaacctgtgaagcatcg

SEQ ID NO: 9 - DNA, 24 bp, Artificial
  Primer
caccatggccgagctgctgcggag

(19) United States
(12) Patent Application Publication
(10) Pub. No.: US 2008/0145883 A1
(43) Pub. Date: Jun. 19, 2008

(54) BIOMARKER IN INFLAMMATORY DISEASES

(76) Inventors: Thomas Baumruker, Wien (AU); Andreas Billich, Wien (AU); Diana Mechtcheriakova, Wien (AU); Alexander Wlachos, Wien (AU)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 11/866,621

(22) Filed: Oct. 3, 2007

(30) Foreign Application Priority Data
Oct. 9, 2006 (EP) 06121954.9

(51) Int. Cl. C12Q 1/42

(52) U.S. Cl. 435/21

(57) ABSTRACT

The use of SPP2 as a target in inflammatory responses, e.g. as a biomarker, e.g. for immune disorders associated with inflammation, such as psoriasis; diagnostic kits comprising means for determining the level of SPP2 and a method for identifying agents that modulates SPP2 activity.

The present invention relates to biomarkers in inflammatory diseases, e.g. the SPP2 gene expression as a player in pro-inflammatory signalling.

Sphingosine-1-phosphate (S1P) is a pleiotropic lipid mediator involved in the regulation of physiological processes including cell growth and survival, cell trafficking and vascular development, vascular tone control, and cardiac functions. In addition, accumulating evidence suggests that S1P may participate in various pathological conditions including angiogenesis, vascular permeability, cancer, inflammation, transplant rejection, and myocardial infarction. S1P is formed from sphingosine (Sph) by two sphingosine kinases (SPHK1 and SPHK2); it is degraded either via irreversible cleavage by S1P lyase (SPL) or via dephosphorylation by phosphatases, e.g. including the S1P-specific phosphohydrolases SPP1 and SPP2. SPP2 as used herein is to be understood as sphingosine-1-phosphate phosphatase 2, also designated as SGPP2, or Spp2, see e.g. C. Ogawa et al, J. Biol. Chem. 278 (2002) 1268-1272 and S. M. Mandala et al, PNAS 97 (2000) 7859-7864; and the broad specificity lipid phosphohydrolases (LPP1 to 3), see e.g. R. Roberts et al, J. Biol. Chem. 273 (1998) 22059-22067.

We have now surprisingly found that the expression of SPP2 is enhanced in cells stimulated with agents, such as TNF-α and LPS, and that SPP2 is involved in IL-1β production by endothelial cells which establishes an unexpected role of SPP2 in the potentiation of cytokine-mediated inflammatory responses. Surprisingly, additionally we have found SPP2 expression to be elevated in lesions of patients with inflammatory immune diseases, e.g. in psoriasis patients, compared with healthy skin, e.g. of such patients. In view of that it can reasonably be expected that SPP2 also plays a role as a mediator in inflammatory immune diseases.

In several aspects the present invention provides
1. SPP2 for use, e.g., or the use of SPP2
1.1 as a target in inflammatory responses;
1.2 for diagnosing disorders mediated, e.g. associated with, e.g. driven by, high levels of SPP2, e.g. or SPP2 activity;
1.3 for diagnosing inflammatory, autoimmune disorders, such as psoriasis.

Disorders as used herein include diseases.

Preferably a disorder is mediated, e.g. associated with, e.g. driven by, high levels of SPP2.

SPP2 as used herein is to be understood as sphingosine-1-phosphate phosphatase 2. “High levels” as used include an elevated, e.g. significantly elevated, level of SPP2 in a sample from a place of an individual where a disease (disorder) may be suspected, such as a sample from an inflammed place, compared with the level of SPP2 in a sample from a healthy donor, or in a sample from a place of said individual, which is believed to be healthy; e.g. in case of psoriasis a sample may be taken from lesional skin and a sample for comparison from non-lesional skin.

Disorders, e.g. including diseases, mediated, e.g. associated with, e.g. driven, by, e.g. high levels of, SPP2 activity are believed to include immune, such as immune, e.g. autoimmune disorders, inflammatory disorders, allergic disorders, infectious diseases, cardiovascular disorders, cancer, disorders associated with transplantation, neurodegenerative disorders; more specifically

disorders such as allergic and nonallergic asthma, chronic obstructive pulmonary disease (COPD), allergic rhinitis, anaphylaxis, allergic gastrointestinal disease, atopic dermatitis, rheumatoid arthritis, psoriasis and other allergic, autoimmune and inflammatory diseases; immunological disorders including arthritis, asthma, multiple sclerosis, immunodeficiency diseases such as AIDS, renovascular hypertension, a disease closely linked to atherosclerosis, diabetes and renal failure, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, autoimmunity disorders, such as autoimmune infertility, lense tissue injury, demyelination, systemic lupus erythematosis, drug induced hemolytic anemia, Sjogren's disease, and scleroderma, cardiovascular disorders, dermatological disorders, metabolic diseases, cancer disorders, e.g. leukemia, gastrointestinal and liver diseases, hematological disorders, reproduction disorders, diseases of the endocrine system, inflammatory diseases, muscle-skeleton disorders, neurological disorders, urological disorders, respiratory diseases, disorders associated with infections such as bacterial, fungal, protozoan, and viral infections, particularly those caused by HIV viruses, cardiovascular diseases including acute heart failure, hypotension, hypertension, angina pectoris, myocardial infarction, hematological diseases, genito-urinary diseases including urinary incontinence and benign prostate hyperplasia, osteoporosis, neurodegenerative disorders, such as peripheral and central nervous system disorders including pain, Alzheimer's disease and Parkinson's disease, metabolic diseases, gastro-enterological diseases, diseases of muscles or the skeleton, immunological diseases, developmental diseases or diseases of the reproductive system, e.g. diseases associated with kidney, brain, heart, intestine, joints, liver or lung disorders.

Disorders and diseases for which SPP2 may be used according to the present invention include preferably

immune, e.g. autoimmune, disorders such as immune disorders associated with inflammation, e.g. psoriasis, rheumatoid arthritis, multiple sclerosis, lupus, e.g. systemic lupus erythematosis, inflammatory bowel disease, autoimmune hepatitis, fibrosis; such as psoriasis, rheumatoid arthritis, multiple sclerosis, lupus, e.g. systemic lupus erythematosis, inflammatory bowel disease;
inflammatory and allergic disorders including atopic dermatitis, asthma, cardiovascular disorders,
disorders or diseases associated with transplantation, disorders or diseases associated with cancer,
more preferably aimmune disorders associated with inflammation, e.g. psoriasis, rheumatoid arthritis, multiple sclerosis, lupus, e.g. systemic lupus erythematosis, inflammatory bowel disease; and/or disorders or diseases associated with transplantation;
e.g. psoriasis, e.g. including conditions which may arise in a patient because of suffering from psoriasis, such as psoriatic arthritis.

Psoriasis is an autoimmune disorder which is related with Th1 cell activation and which is associated with inflammatory skin disease in which skin cells replicate at an extremely rapid rate. New skin cells are produced about 8 times faster than normal—over several days instead of a month—but the rate at which old cells slough off remains unchanged. This causes cells to build up on the skin's surface, forming thick patches, or plaques, of red scores (lesions) covered by flaky, silvery-white dead skin cells (scales).

Rarely life-threatening, at its mildest, psoriasis can be itchy and sore. At its worst, it's painful, disfiguring and debilitating. About ⅔s of the people with psoriasis have a mild form of the disease. About ⅓ have moderate or severe psoriasis. Psoriasis can affect people at any age, but it most often strikes those between the ages of 15 and 35.

There are 5 forms of psoriasis. Plaque psoriasis is the most common—affecting 4 out of 5 people who have psoriasis. Plaque psoriasis may start with small reed bumps and progress to larger lesions.

The plaques of psoriasis occur most frequently on the elbows, knees, other parts of the legs, scalp, back, face, palms and sole of the feet. Psoriasis can also affect the fingernails and toenails, causing pitting, discoloration or tissue buildup around the nails.

According to the National Institute of Arthritis and Musculoskeletal and Skin Diseases, about 15% of people with psoriasis also get psoriatic arthritis, which can be progressively disabling if untreated.

It is believed that T lymphocytes (T cells) play an important role in psoriasis and it was found that Th1 cell activation plays a major role. Psoriasis also has a genetic component: in about ⅓ of psoriasis cases, there is a family history of the disease.

T cells circulate throughout the body, orchestrating the immune system's response to foreign invaders like bacteria or viruses. In people with psoriasis, the defective T cells are overactive and migrate to the skin as if to heal a wound or ward of an infection. This process leads to the rapid growth of skin cells, triggering inflammation and development of lesions. Until now, no single test exists to diagnose psoriasis, but a dermatologist can usually determine it by appearance of the skin and by locking at an individual's personal and family medical history.

A dermatologist can usually determine psoriasis by appearance of the skin and by locking at an individual's personal and family medical history, but, until now, no single test exists to diagnose psoriasis.

In several other aspects the present invention further provides
2. SPP2, e.g. or the use of SPP2, as a biomarker, for a use as indicated under 1.1 to 1.3 above
   e.g. in a sample of an individual,
   e.g. in a sample of a body fluid or a tissue sample of an individual,
   e.g. in a biopsy-sample of an individual,
   e.g. skin biopsy-sample, of an individual,
   e.g. which sample is originating from a patient suffering from a disease, e.g. originating locally from the place where a disease may be suspected, e.g. originating from a skin biopsy, e.g. originating from plaques, of a patient which is believed to suffer from psoriasis.

SPP2 as indicated under any of 1. or 2 above includes SPP2, e.g. in dendritic cells, in any form, e.g. in the form of
a nucleic acid encoding SPP2, e.g. including a nucleic acid encoding a derivative of SPP2,
SPP2 protein, e.g. including protein which is a SPP2 derivative, or
SPP2 secreting cells, e.g. or a derivative of SPP2 secreting cells.

“A derivative” of SPP2 nucleic acid or protein, e.g. in secreting cells, according to the present invention includes a fragment, a mutant, a variant, an homolog or a modification of a SPP2 protein, or of a nucleic acid encoding SPP2, which retains, e.g. essentially, the biological function of GPR91, e.g. which retains, e.g. essentially, the biological function of SPP2, e.g. in dendritic cells.

SPP2-secreting cells, e.g. including SPP2 producing cells, include antigen presenting cells (APC), such as dendritic cells (DC).

Thus, SPP2 for use as provided by the present invention includes splice variants encoded by mRNA generated by alternative splicing of a primary transcript, amino acid mutants, posttranslational modifications, such as glycosylation and phosphorylation variants, and modifications which are covalent derivatives of SPP2 and which retain the biological function of SPP2, e.g. in dendritic cells. Exemplary SPP2 derivatives include modifications wherein the SPP2 protein is covalently modified by substitution, e.g. substitution originating from appropriate means, e.g. chemical or enzymatic means, by a moiety in the SPP2 protein. Such a moiety e.g. includes one or more amino acids, e.g. naturally occurring amino acids and other than naturally occurring amino acids, and/or a detectable moiety. A detectable moiety includes an enzyme, a radioisotope, tags, toxins and genes such as oncogenes and tumour suppressor genes. SPP2 derivatives further include naturally occurring variants of SPP2, e.g. provided within a particular species. Such a variant may be encoded by a related gene of the same gene family, by an allelic variant of a particular gene, or represent an alternative splicing variant of the SPP2 gene.

A SPP2 derivative as used herein also includes fragments of a nucleic acid encoding SPP2, or of the SPP2 protein, and comprises individual SPP2 domains and smaller polypeptides derived from SPP2 domains. Preferably, smaller polypeptides derived from SPP2 according to the invention define a single functional activity which is characteristic of SPP2. Fragments may in theory be of almost any size, as long as they retain the biological characteristic of SPP2. Preferably, fragments will be between 12 and 210 nucleic acids in length or between 4 and 70 amino acids, respectively. Longer fragments are regarded as truncations of the full-length SPP2.

Derivatives of SPP2 as used herein also comprise mutants thereof, which may contain amino acid deletions, additions or substitutions, subject to the requirement to retain the biological function of SPP2, e.g. in dendritic cells. Conservative amino acid substitutions may be made substantially without altering the nature of SPP2, e.g. by truncations from the 5′ or 3′ ends. Deletions and substitutions also include deletions and substitutions in fragments of SPP2. SPP2 mutants may be produced from a DNA encoding SPP2 which has been subjected to in vitro mutagenesis resulting e.g. in an addition, exchange and/or deletion of one or more amino acids in SPP2. For example, substitutional, deletional or insertional variants of SPP2 can be prepared by recombinant methods and screened for functional similarity to the native forms of SPP2.

Derivatives of SPP2 as used herein also include SPP2 homologs, preferably SPP2 homologs retain substantial homology with SPP2. As used herein, “homology” means that SPP2 and a SPP2 homolog share sufficient characteristics to retain the biological function of GPR91 in dendritic cells. Preferably, homology is used to refer to sequence identity. Thus, the derivatives of SPP2 preferably retain substantial sequence identity with the nucleic acid sequence as given in

“Substantial homology”, where homology indicates sequence identity, means more than 50% sequence identity, preferably more than 75% sequence identity and even more preferably a sequence identity of 80% and more, e.g. 90% and more, such as 90%, 91%, 92%, 93%, 94%, 95%, 96%, 97%, 98% or 99%.

Preferably SPP2 is originating from a mammal.

The nucleic acid encoding SPP2 preferably has the nucleic acid sequence as disclosed for SPP2. The SPP2 protein preferably corresponds to the translated protein sequence of the above mentioned nucleic acid.

Biomarker as used herein means that determination (=detection and/or quantification) of elevated levels of SPP2 in a sample, molecule in a sample of an individual is an indicator for a disorder or disease as such and/or is useful for monitoring the status of a disorder or disease related with SPP2, e.g. with the level of SPP2 and, in consequence, with SPP2 activity.

In another aspect the present invention provides a method for diagnosing a disorder or disease which is mediated, e.g. associated with, e.g. driven by, elevated SPP2 levels, e.g. or SPP2 activity, comprising
a) providing a sample of an individual, e.g. which sample is originating locally from the place where a disease may be suspected, e.g. originating from plaques of a patient which is believed to suffer from psoriasis;
b) determining the level of SPP2 in said sample,
c) comparing the level of SPP2 as determined in step b) with a reference level, e.g. with the level of SPP2 from a sample which originates from a healthy donor, e.g. or which originates from a place of said individual which is suspected to be healthy, e.g. with a healthy skin biopsy from said individual, and
d) diagnosing a disorder or disease, if the SPP2 level in the sample is, e.g. significantly, elevated compared with the SPP2 reference level, e.g. the level in sample of a healthy donor, or, the level in a sample from a place of said individual which is suspected to be healthy,
   e.g. which disorder or disease is mediated, e.g. associated with, e.g. driven by, elevated levels of SPP2, e.g. or by SPP2 activity.

In another aspect the present invention provides a method for monitoring the therapeutic efficacy in the treatment of an individual with a substance which is expected to have an effect on reducing or curing a disorder or disease which is mediated, e.g. associated with, e.g. driven by, elevated SPP2 levels, e.g. or SPP2 activity, which method comprises determining the level of SPP2 in dendritic cells in a sample of said individual suffering from such disease and comparing the level determined with the level of SPP2 prior to administration of said substance.

A sample of an individual according to a use or a method of the present invention includes a sample of a body fluid or a tissue sample. A body fluid may be derived e.g. from blood, e.g. including isolated mononuclear cells, or from a blood fraction, e.g. including plasma or serum, preferably serum. A tissue sample may be a biopsy, e.g. such as a skin biopsy.

In another aspect the present invention provides the a use or a method of the present invention wherein a sample is a body fluid or a tissue sample of an individual, e.g. a body fluid may be derived from blood, e.g. isolated cells, such as dendritic cells, or from a blood fraction, e.g. plasma or serum, e.g. serum; e.g. the tissue sample may be a biopsy, e.g. such as a skin biopsy.

Cells, e.g. dendritic cells from a sample of an individual may be isolated as appropriate, e.g. according, e.g. analogously, to a a method as conventional.

Detection means in cells for determining the level of SPP2 include means as conventional, e.g. immunoassays, such as an immunodiagnostic method, an enzyme linked immunoassay (ELISAs); a fluorescence based assay, such as dissociation enhanced lanthanide fluoroimmunoassay (DELFIA), an radiometric assay or by carrying out a SPP2 specific Polymerase Chain Reaction (PCR); specifically detection means include a molecule which specifically recognizes SPP2, e.g. a molecule which is directly or indirectly detectable, preferably comprising an antibody, including antibody derivatives or fragments thereof, e.g. an antibody which recognizes SPP2, e.g. a label bearing SPP2 recognizing antibody. Such label may be a conventional label, e.g. biotin or an enzyme such as alkaline phosphatase (AP), horse radish peroxidase (HRP) or peroxidase (POD) or a fluorescent molecule, e.g. a fluorescent dye, such as e.g. fluorescein isothiocyanate. Preferably the label is biotin. The label bearing molecule, e.g. the label bearing antibody, may be detected according to methods as conventional, e.g. via fluorescence measurement or enzyme detection methods.

An antibody fragment or antibody derivative includes a fragment or a derivative, e.g. chemically or enzymatically modified, of an antibody which still is capable of recognising SPP2.

SPP2-secreting cells in a sample of a body fluid of an individual, e.g. blood, may be determined by a method as conventional, e.g. by the following method:

Cells, e.g. fendritic cells may be purified, e.g. separated by a density gradient, from the sample, e.g. blood, and the purified cells obtained are stained. Anti-SPP2 antibodies, e.g. fluorescence labeled anti-SPP2 antibodies, are added to the stained cell preparation, optionally after stimulation of the cells, e.g. with interleukin-4, and the level of SPP2-secreting cells is determined.

Optionally, SPP2 comprised in the sample or the SPP2 recognizing, e.g. detectable, molecule comprised in the detection means is immobilized on a solid phase. An appropriate solid phase includes e.g. conventional solid phases used for immobilization, e.g. a plastic plate like a polystyrene or polyvinyl plate, especially a microtiter plate. Also microbeads can be used as a solid phase, e.g. coated microbeads. The solid phase can be coated with a coating material the nature of which depends e.g. on the label comprised in the detection means. The coating material should be able to bind to the label, e.g. if the label is biotin a coating material includes streptavidin, e.g. covalently bound to the solid phase. Preferably determination of SPP2 in dendritic cells is carried out by using a molecule which specifically recognizes the SPP21, e.g. an antibody, an antibody derivative, or an antibody fragment, such as an anti SPP2 antibody, e.g. a commercially available SPP2 specific antibody. Detection of SPP2-antibody formation preferably is carried by an immunodiagnostic assay method.

In another aspect the present invention provides a method for diagnosing a disorder or disease which disorder or disease is mediated, e.g. associated with, e.g. driven by, elevated levels of SPP2, e.g. or by SPP2 activity,

wherein the level of SPP2 in cells, e.g. dendritic cells, is determined by use of an SPP2 specific antibody.

In another aspect the present invention provides

a kit for diagnosing of a disorder or disease which disorder or disease is mediated, e.g. associated with, e.g. driven by, elevated levels of SPP2, e.g. or by SPP2 activity, comprising
a) means for detecting the level of SPP2 in a sample, e.g. a molecule which recognizes SPP2, and which molecule optionally is in a labeled form,
b) instructions how to use said kit, e.g. in in dendritic cells,
c) optionally detection means,
d) optionally a solid phase; and
   A method of providing such kit according to the present invention by providing a), b) and optionally c) and/or d), for use in the diagnosing of a disorder which disorder is mediated, e.g. associated with, e.g. driven by, elevated levels of SPP2, e.g. or by SPP2 activity Such kit may further comprise a substantial component, e.g. including an appropriate environment of a sample to be tested and, e.g. appropriate means to determine SPP2 in a sample to be tested.

In a further aspect the present invention provides an assay for identifying an agent that mediates a disorder which is mediated by elevated SPP2 levels or SPP2 activity, comprising
a) determining the level of SPP2 in dendritic cells of a sample of an individual, in the absence and in the presence of a candidate compound which may be expected to modulate the level of SPP2,
b) identifying a candidate compound which modulates the level of SPP2 as determined in step a) as an agent, e.g. and
c) using such agent as a pharmaceutical in the treatment of disorders or diseases mediated, e.g. associated with, e.g. driven, by SPP2 activity.

Preferably a candidate compound identified decreases the level of SPP2.

The level of SPP2 is determined as appropriate, e.g. as described herein.

A candidate compound as described herein is a compound which may be expected to modulate the level of SPP2, or SPP2 activity or SPP2 secreting cells, and includes compound(s) (libraries) from which its influence on SPP2 can be determined. Compound (libraries) include for example oligopeptides, polypeptides, proteins, antibodies, mimetics, small molecules, e.g. low molecular weight compounds (LMW's).

An agent is a candidate compound which modulates the level of the level of SPP2, or SPP2 activity or SPP2 secreting cells, e.g. in cells, such as dendritic cells in a sample form a patient, e.g. a blood sample, such as serum, or a skin biopsy. An agent includes oligopeptides, polypeptides, proteins, antibodies, mimetics, small molecules, e.g. low molecular weight compounds (LMW's).

In another aspect the present invention provides an agent identified by an assay or a method of the present invention.

An agent of the present invention may exhibit pharmacological activity and is therefore useful as a pharmaceutical. An agent of the present invention may show therapeutic activity, e.g. in disorders or diseases mediated, e.g. associated with, e.g. driven by SPP2 activity.

In another aspect the present invention provides the use of an agent of the present invention as a pharmaceutical in disorders mediated, e.g. associated with, e.g. driven by SPP2 activity.

For pharmaceutical use an agent of the present invention for treatment includes one or more, preferably one, agent of the present invention, e.g. a combination of two or more agents of the present invention.

In another aspect the present invention provides the use of an agent of the present invention for the manufacture of a medicament for the treatment of disorders or diseases mediated, e.g. associated with, e.g. driven by SPP2 activity.

In another aspect the present invention provides a pharmaceutical composition comprising an agent of the present invention beside at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a method for the treatment of disorders or diseases mediated, e.g. associated with, e.g. driven, by SPP2 activity, comprising administering an effective amount of an agent of the present invention to a subject in need of such treatment.

For such treatment, the appropriate dosage will, of course, vary depending upon, for example, the chemical nature and the pharmakokinetic data of a compound of the present invention used, the individual host, the mode of administration and the nature and severity of the conditions being treated. However, in general, for satisfactory results in larger mammals, for example humans, an indicated daily dosage includes a range
from about 0.001 g to about 1.5 g, such as 0.001 g to 1.5 g;
from about 0.01 mg/kg body weight to about 20 mg/kg body weight, such as 0.01 mg/kg body weight to 20 mg/kg body weight,
   for example administered in divided doses up to four times a day.

An agent of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration; via medical devices for local delivery,

e.g. stents,
e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

For topical use, e.g. including administration to the eye, satisfactory results may be obtained with local administration of a 0.5-10%, such as 1-3% concentration of active substance several times daily, e.g. 2 to 5 times daily.

An agent of the present invention may be administered in the form of a pharmaceutically acceptable salt, e.g. an acid addition salt or metal salt; or in free form; optionally in the form of a solvate. An agent of the present invention in the form of a salt may exhibit the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

### DESCRIPTION OF THE FIGURES

FIG. 1

Shows the expression of TF mRNA, SPP1 mRNA, SPP2 mRNA, SPHK1 mRNA and SPHK2 mRNA in primary endothelial cells, HUVEC upon cell activation with TNF-alpha. Shows strong upregulation of mRNAs encoding tissue factor (TF) which confirms HUVEC activation by TNF-alpha and shows that the expression of SPP2 mRNA is strongly induced in primary endothelial cells, HUVEC, upon cell activation with TNF-alpha, e.g. in contrast to mRNA of the other enzymes SPP1, SPHK1 and SPHK2.

HUVEC are stimulated by TNF-α (100 U/ml) for up to 24 hrs. Relative expression is calculated using the ΔΔC_{T} method. Levels of mRNA are normalized to an average of HKGs (β2M, UBC, EF1A) and expressed relative to unstimulated cells (time point “0”). Data are means ±S.E.M. of six independent experiments with cells from different donors, each performed in duplicate; *p<0.05, **p<0.01, ***p<0.001, n.s., not significant by t test compared to unstimulated cells.

FIG. 2

Shows that both, SPHK1 and SPP2 are triggered in neutrophils upon stimulation by LPS Freshly isolated neutrophils are stimulated with LPS (500 ng/ml) for 0.5 h, 1 h, 2.5 h, and 6 h. Relative expression is calculated using the ΔΔC_{T} method. Levels of mRNA are normalized to an average of HKGs and expressed relative to unstimulated cells (time point “0”). Results are means ±S.E.M. of four independent experiments with cells from different donors, each performed in duplicate, *p<0.05, **p<0.01, n.s., not significant by t test compared to unstimulated cells.

FIG. 3

### SPHK and Phosphatase In Vitro Activity Assays

(A) HUVEC are collected at 0, 0.25 h, 2.5 h, 6 h, and 7.5 h after stimulation with TNF-alpha. (B) Neutrophils are collected at 0, 0.25 h, 2.5 h, 6 h, and 7.5 h after stimulation with LPS. In lysates of the cells, SPHK activity is measured using sphingosine as substrate in buffers favoring either SPHK1 or SPHK2 activities as described in the Examples. Phosphatase activity is measured using [3-³H]-S1P as a substrate. To check effects of NaF and Triton X-100 on induced phosphatase activity, lysates prepared from cells stimulated for 7.5 h are incubated with [3-³H]S1P in the presence of 0.1% Triton X-100 (filled square) or 5 mM NaF (open triangle). The specific enzyme activity is given as pmol of product formed per minute per milligram of total protein. Data are means ±S.D. for four separate experiments performed with different donors, *p<0.05, **p<0.01 by t test.

(B) SPHK activity assay with neutrophil lysates after 5 min, 10 min, and 15 min of LPS stimulation. Values are given relative to unstimulated cells (time point “0”) ±S.D. (n=3).

FIG. 4 and FIG. 5

### Identification of NFkappaB Binding Sites Within the Proximal SPP2 Promoter

FIG. 4

(A) Radioactively labeled oligonucleotides spanning the promoter regions around −753 (OL_{SPP2/-753}) and −853 (OL_{SPP2/-853}) are incubated with nuclear extracts from unstimulated HUVEC (lane 1) and HUVEC treated with TNF-alpha for 1 h (lanes 2 to 8). The TNF-alphy-induced NFkappaB complex is indicated by an arrow. TNF-alpha-stimulated extracts are analyzed by competition with a 50-fold molar excess of unlabeled oligonucleotides: self (lane 3), non-specific/control Sp1 (lane 4), NFkappaB consensus (lane 5). Supershift experiments are performed using anti-p65/RelA (lane 6), anti-cRel (lane 7), and non-specific/control anti-EGR-1 (lane 8) antibodies. Shown is one experiment that is representative of two performed with similar results.

FIG. 5

(B) Silencing of NFkappaB/RelA by siRNA prevents TNF-alpha-triggered up-regulation of SPP2. HUVEC are transfected with RelA siRNA or scrambled siRNA (100 nM each). 24 h post-transfection cells are stimulated with TNF-alpha and at indicated time points collected for RNA isolation. Levels of RelA, SPP2, and VCAM-1 mRNAs are normalized to an average of HKG values and expressed relative to unstimulated cells (time point “0”) transfected with scrambled siRNA. Data are means ±S.E.M. of three independent experiments, each performed in duplicate, *p<0.05, **p<0.01 by t test. Inset, Western blot analysis with anti-RelA antibodies of whole HUVEC cell lysates harvested 24 h after transfection with scrambled siRNA (lane 1) and RelA (lane 2) siRNAs. Membrane is reprobed with anti-actin antibodies to ensure equal loading of samples. Shown is one experiment representative of three performed with similar results.

FIGS. 6, 7 and 8

Show that SPP2-specific siRNA significantly reduces the TNF-alpha-triggered expression of IL-1beta.

HUVEC ae transfected with SPP2 siRNA or scrambled siRNA (150 nM each). To silence SPP2, two siRNAs directed to different regions are combined at 75 nM each; transfection with each siRNA duplex separately results in about 60% reduction of SPP2 mRNA levels (data not shown). 24 h post-transfection cells are stimulated with TNF-alpha (100 U/ml) at indicated time points collected for RNA isolation followed by real-time RCR analysis or for Western blot. (FIGS. 6 (A), 7 (C), 8 (E)). Relative expression of target genes after TNF-alpha stimulation is calculated using the ΔΔC_{T} method. Levels of mRNA are normalized to an average of HKG values and expressed relative to unstimulated cells (time point “0”) transfected with scrambled siRNA. Data are means ±S.D. (n=3).

FIG. 6

In vitro phosphatase assay with lysates from SPP2 siRNA- and scrambled siRNA-transfected HUVEC after 4 h, 6 h, and 8 h of stimulation by TNF-α. Y-axis: values are given relative to unstimulated cells (time point “0”) ±S.D. from three independent experiments.

FIG. 6 (B)

EGFP-SPP fluorescence in HUVEC transfected with

(a) control siRNA together with EGFP-SPP2,
(b) SPP2 siRNA together with EGFP-SPP2,
(c) control siRNA together with EGFP-SPP1,
(d) SPP2 siRNA together with EGFP-SPP1. Representative images of three independent experiments are shown.
(e) EGFP-positive cells are counted. Results are shown as % of positive cells in presence of control siRNA and represent means ±S.D. from three independent experiments.

FIG. 7 (D)

Western blot analysis of whole HUVEC cell lysates with antibodies directed to IL-1 precursor. Cells transfected with siRNAs are harvested at 0 h, 16 h, and 24 h time points of stimulation with TNF-α. An arrow indicates the position of 31/33 kDa IL-1β precursor. Re-probing the same membrane with anti-actin antibodies is used to ensure equal loading. Results are representative of four independent experiments.

FIG. 9

Shows increased expression of SPP2 mRNA in lesional skin from psoriasis patients. Skin biopsies, namely uninvolved non-lesional (NL) and lesional from the same patient, from five psoriatic patients are used for total RNA preparation. Relative expression is calculated using the ΔΔC_{T} method. Data are corrected to an average of HKG expression values obtained for each sample and calculated relative to the NL sample of patient 1. A line connects the paired samples taken from each patient's NL and psoriatic skin. [symbol] indicates the mean value for each type of specimen.

According to the present invention the following surprising findings are made:

### 1. SPP2 Expression is Induced in Endothelial Cells Stimulated by TNF-alpha

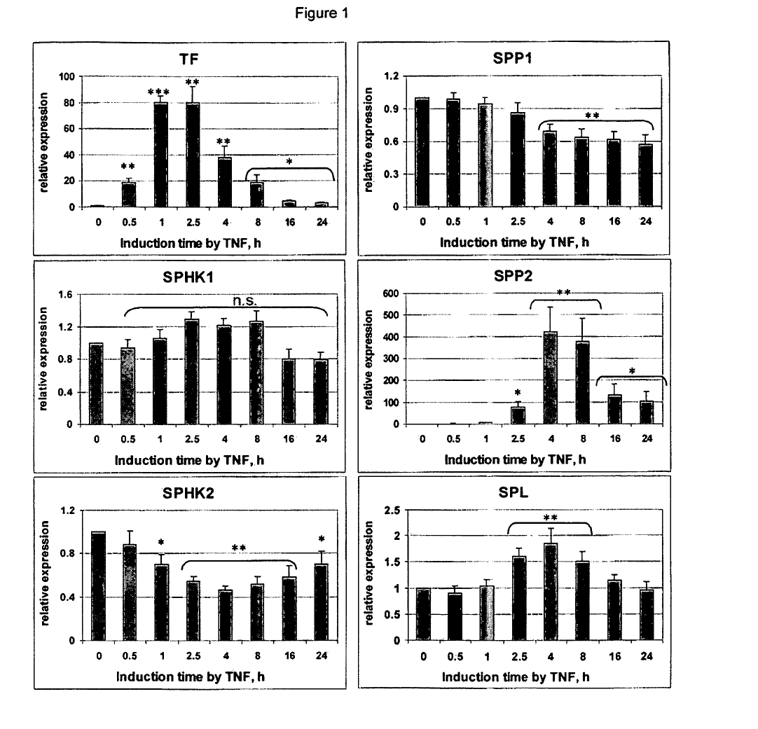

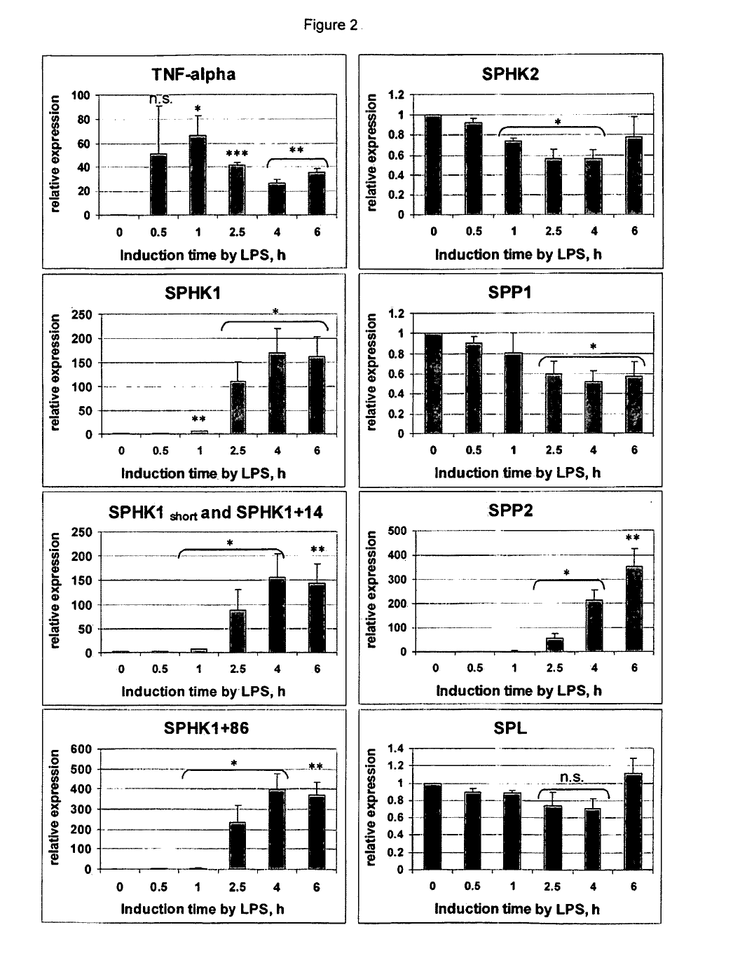

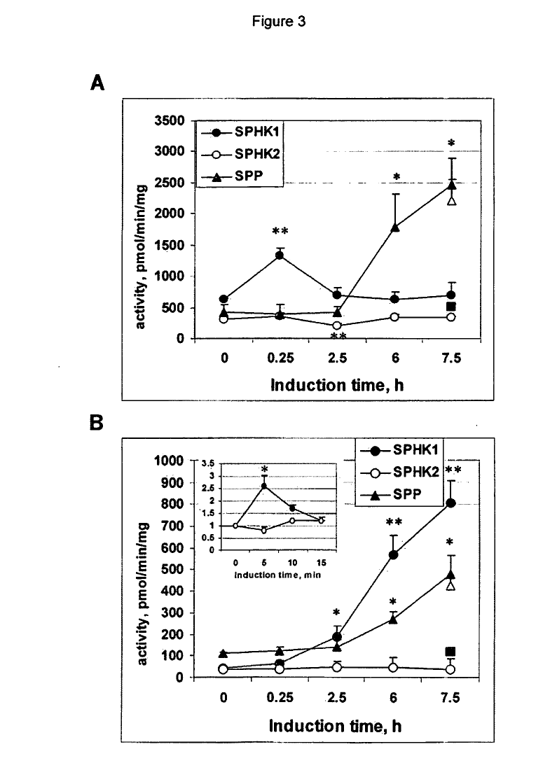

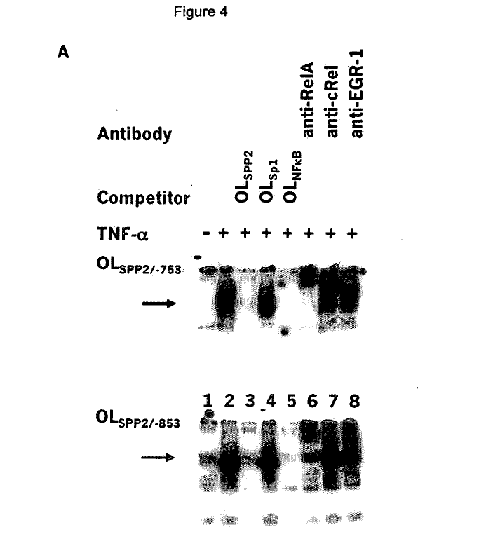

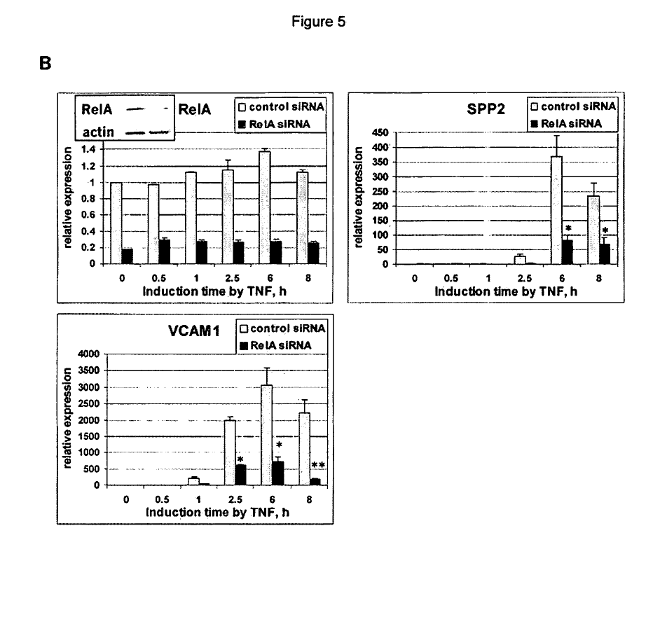

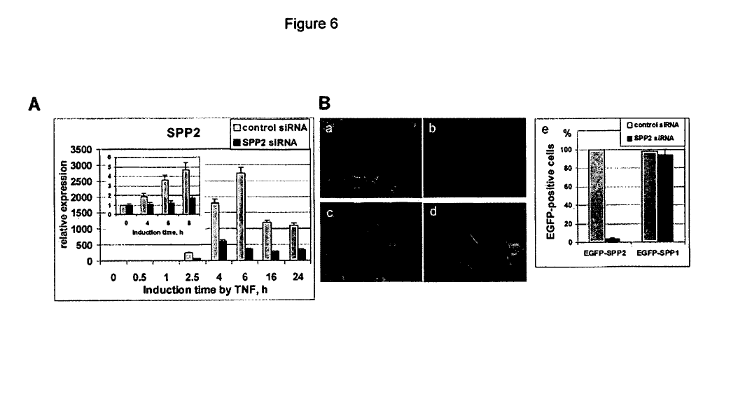

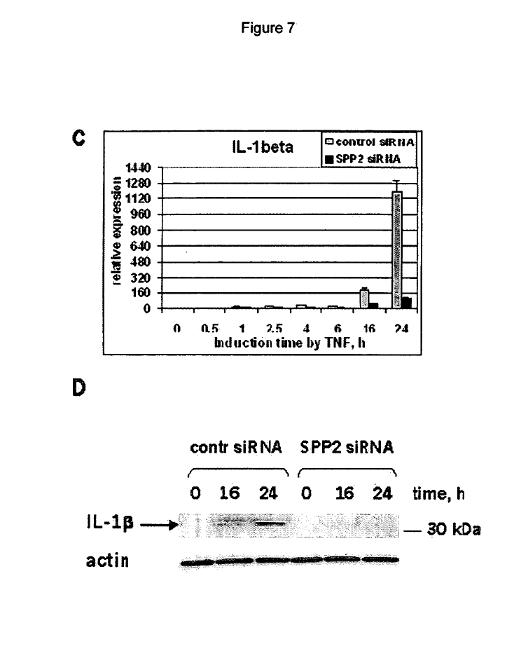

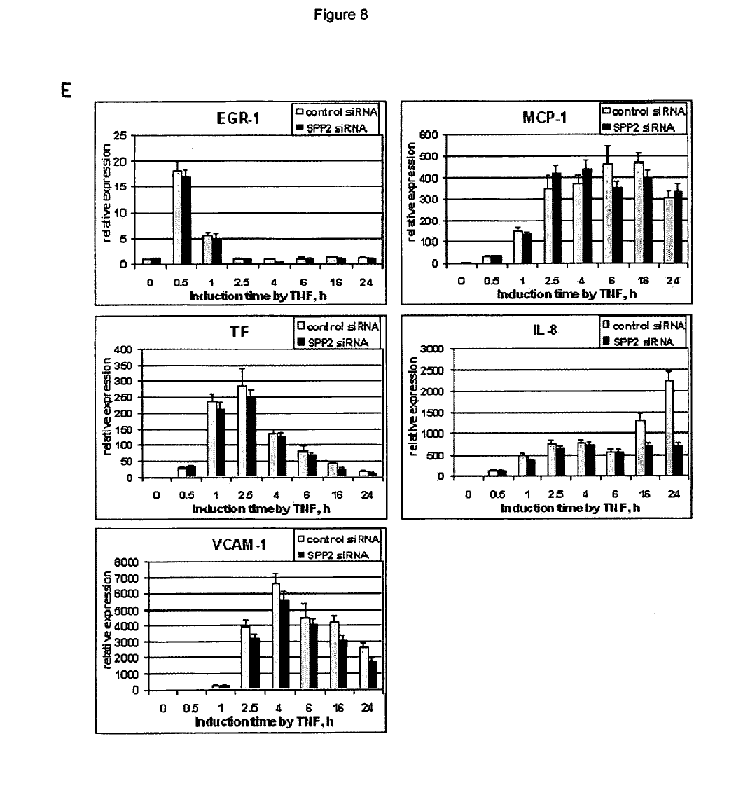

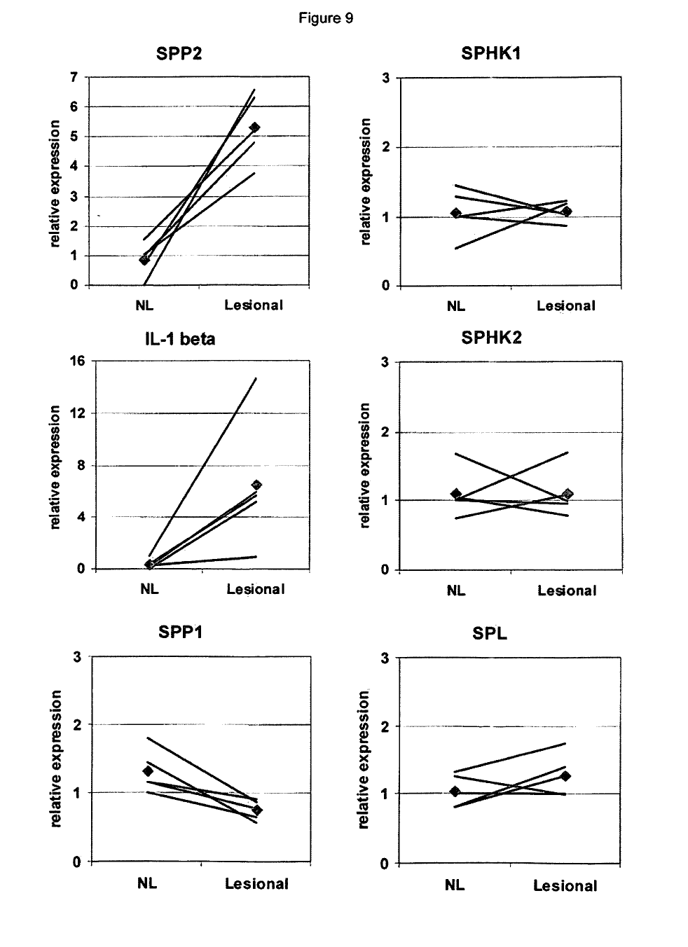

The expression patterns of enzymes involved in S1P metabolism in endothelial cells by real-time PCR has been examined. HUVEC from six different donors are stimulated with TNF-alpha over a period of 24 h. Activation of HUVEC is confirmed by the strong up-regulation of the mRNAs encoding tissue factor (TF) (FIG. 1) and ICAM1 (data not shown). Under normal culture conditions, HUVEC does not express SPP2, while SPP1 is readily detected. Upon treatment with TNF-alpha, SPP2 mRNA is strongly induced with maximal expression measured at 4 h of stimulation followed by a decline (FIG. 1). In contrast to SPP2, SPP1 expression levels are reduced upon activation of the cells, but not more than by a factor of 2. SPL mRNA levels are slightly increased (factor of about 2) at around 4 h after stimulation. All splice variants of SPHK1 and 2 (see e.g. C. Ward et al, J. Biol. Chem. 274 (1999) 4309-4318) are detectable in unstimulated HUVEC (see FIG. 1 and TABLE 1 for primer localization and sequence). For SPHK1, no significant modulation of expression levels is detected. Moderate reduction in SPHK2 total mRNA levels (by a factor of around 2) (Figure) is detected to a similar extent both for SPHK2_{long} and SPHK2ₛₕₒᵣₜ (data not shown). Thus, the most pronounced effect of TNF-alpha stimulation observed here is the strong, transient up-regulation of SPP2.

### 2. SPP2 and SPHK1 are Triggered in Neutrophils Stimulated by LPS

Freshly isolated neutrophils from four donors ae exposed to LPS and the mRNA levels of the five enzymes involved in S1P metabolism ae studied over a period of 6 h. Strong induction of TNF-alpha (FIG. 2), IL-6, IL-8, and MCP1 mRNA levels (data not shown) confirms activation. Similar to TNF-alpha-stimulated HUVEC, pronounced induction of SPP2 mRNA starting at 2.5 h of stimulation is detected, with maximal increase at 6 h (FIG. 2). SPP1 mRNA is modestly down-regulated (factor of about 2) similar to findings in HUVEC. SPL mRNA levels are not changed significantly.

In contrast to the situation in TNF-alpha-stimulated HUVEC, in addition to SPP2, total SPHK1 mRNA levels are also significantly enhanced in the LPS-stimulated neutrophils (FIG. 2); induction with similar kinetics is observed for all of the SPHK1 splice variants. SPHK2 is not induced in these cells; rather a slight reduction in expression is seen.

### 3. Up-Regulation of SPP2 and SPHK1 Enzyme Activity in Endothelial Cells and Neutrophils

We asked whether the transcriptional up-regulation of SPP2 in the TNF-alpha-stimulated HUVEC or SPHK1 and SPP2 in neutrophils upon LPS stimulation results in increased enzymatic activities. Cell lysates are collected at 15 min, 2.5 h, 6 h, and 7.5 h after stimulation. A short-term period is included to the study to be able to detect an early peak of SPHK1 activation which is previously shown to be attributed to posttranslational mechanism(s), see e.g. K. R. Johnson et al, J. Biol. Chem. 278 (2003) 34541-34547; and S. Pitson et al, EMBO J. 22 (2003) 5491-5500. SPHK activity is measured using sphingosine as a substrate with buffer conditions favoring either SPHK1 or SPHK2 activity, see e.g. A. Billich, F. Bornancin, P. Devay, D. Mechtcheriakova, N. Urtz, T. Baumruker, J. Biol. Chem. 278 (2003) 47408-47415. In TNF-α-stimulated HUVEC, an early peak of SPHK1 activation at 15 min is detected (FIG. 3, A); in line with the real-time PCR data, no significant modulation in activity is observed within the next 7.5 h of stimulation. SPHK2 activity is transiently reduced by around 30% at 2.5 h. No modulation in phosphatase activity is seen at 15 min of stimulation (FIG. 3, A); an additional short-term treatment (5 min and 10 min) does not reveal any significant changes (data not shown). However, at late time points after stimulation the phosphatase activity is strongly triggered showing an about 6-fold increase at 7.5 h. Furthermore, this increase in the phosphatase activity is inhibited in the presence of 0.1% Triton X-100, while it is insensitive to 5 mM NaF (FIG. 3, A). Previous studies have demonstrated that Triton X-100, but not NaF, has strong inhibitory effect on SPP2 activity, while the activity of SPP1 could be inhibited by both Triton X-100 and NaF, see e.g. C. Ogawa et al, J. Biol. Chem. 278 (2002) 1268-1272 and S. M. Mandala et al, PNAS 97 (2000) 7859-7864. In contrast, the activity of all LPPs is rather enhanced by Triton X-100 when it is used for appropriate substrate presentation in micelles, see e.g. R. Roberts et al, J. Biol. Chem. 273 (1998) 22059-22067 and J. M. Furneisen et al, Biochim. Biophys. Acta 1484 (2000) 71-82. Therefore, the induced phosphatase activity we recorded in HUVEC extracts can be attributed to SPP2.

In LPS-stimulated neutrophils, two waves of triggered SPHK1 activity are detected: an early activation within 10 min after stimulation with maximal increase of about 2.5 (FIG. 3, B) and a delayed one starting at 2.5 h after stimulation and showing maximally a 19-fold increase at 7.5 h (FIG. 3B). Thus, in line with the real-time PCR data, the activity of SPHK1 is strongly induced in neutrophils in a time-dependent manner. SPHK2 activity does not change significantly over the period chosen. Phosphatase activity is triggered in a time-dependent manner with an about 4-fold increase at 7.5 h time point. Similar to the situation with HUVEC, this increase in the phosphatase activity is inhibited by 95% in the presence of 0.1% Triton X-100, while it is insensitive to 5 mM NaF (FIG. 3B), thus indicating a major contribution of SPP2. No short-term effects of LPS on the phosphatase activity in neutrophils are found.

Of note, the specific activities of SPHK1 and SPHK2 in HUVEC at baseline are 630±14 and 303±41 pmol/min/mg, respectively; the total phosphatase activity is 426±111 pmol/min/mg. The specific activities of SPHK1 and SPHK2 in neutrophils at baseline are 41.7±3.5 and 38.7±2.5 pmol/min/mg, respectively; the phosphatase activity in unstimulated neutrophils is 109±8 pmol/min/mg. Thus, both kinase and phosphatase activities detected in neutrophils are found to be significantly lower than those in HUVEC. Next, the levels of S1P and sphingosine in HUVEC and neutrophils upon stimulation with TNF-α and LPS, respectively have been examined. It was found in HUVEC a transient increase of S1P by about 30% after 15 min of stimulation compared to unstimulated cells concomitant with reduced sphingosine levels (TABLE 1). No significant changes in S1P and sphingosine levels are detected at later time points of cell activation. In neutrophils, a slight time-dependent increase in S1P levels is detected during the 8 h period of stimulation paralleled by reduction of sphingosine levels.

### 4. Transcriptional Regulation of SPP2 Following Stimulation of Various Cell Types

To expand on our finding of SPP2 up-regulation in TNF-alpha-stimulated HUVEC, other stimuli acting on this cell type are used. Both LPS and PMA also induces SPP2 up-regulation, while the mitogenic factor EGF and the angiogenic factor VEGF does not (TABLE 2). LPS but not EGF leads to SPP2 mRNA increase in human neutrophils (TABLE 2). Furthermore, SPP2 message is induced in PMA-stimulated promyelocytic leukemia HL60 cells, cytokine-stimulated lung carcinoma A549 cells, and dendritic cells (DC) during maturation (TABLE 2). Remarkably, activation of neutrophils by both LPS and EGF, stimulation of HL60 cells by PMA, and maturation of monocyte-derived DC in the presence of LPS results in profound increase in SPHK1 mRNA levels, indicating a certain degree of cell-type specificity in the regulation of this gene.

### 5. SPP2 is an NFκB-Dependent Gene

Based on the up-regulation of SPP2 transcription by various inflammatory agents in a cell type-independent manner it was expected that the SPP2 promoter would have NFkappaB binding site(s). Indeed, two potential NFkappaB motifs are found at positions −753 and −853 relative to the translational start site by in silico analysis. Using the oligonucleotides spanning the SPP2 promoter regions around −753 (OL_{SPP2/-753}) and −853 (OL_{SPP2/-853}) and nuclear extracts from the TNF-alpha-stimulated HUVEC, it was confirmed the prediction of the NFkappaB binding sites (FIG. 4A). TNF-alpha induces the formation of a strong nucleoprotein complex, which is blocked by an excess of unlabelled OL_{SPP2/-753} or OL_{SPP2/-853} (lane 3) as well as by an oligonucleotide harboring a consensus NFκB site (OL_{NFκB}), but not by an oligonucleotide carrying an SP1 binding site (OL_{Sp1}). Furthermore, the induced complex is supershifted by antibodies against the NFkappaB/RelA subunit (lane 6). Pre-incubation with anti-cRel antibodies has only little effect on the complex formation with OL_{SPP2/-753} suggesting a minor contribution of this NFkappaB family member in the protein-DNA complex. No supershift is observed with the cRel antibody on the complex with the OL_{SPP2/-853} probe. Similar results are obtained with the control anti-EGR1 antibody.

To assess the functional involvement of the NFkappaB/RelA subunit in SPP2 promoter regulation, we used siRNA directed against RelA. Silencing by RelA siRNAs of RelA mRNA and protein levels (FIG. 5, B) results in a significant inhibition (by about 80%) of the TNF-alpha-triggered SPP2 mRNA, indicating critical involvement of NFkappaB/RelA in induction of SPP2 transcription. NFkappaB-dependent VCAM-1 expression is reduced by RelA siRNAs to a similar extent (FIG. 5, B).

Analysis of the major SPHK1 promoter elements reveals clear distinctions with the organization of the SPP2 promoter; based on the same algorithm, no NFkappaB motifs are identified (data not shown).

6. Effect of SPP2 Silencing on the TNF-Alpha-Induced Expression of IL-1beta in HUVEC

To explore a possible biological consequence of SPP2 induction, the effects of SPP2-specific siRNA in HUVEC are examined. Based on the kinetics of SPP2 induction in response to TNF-alpha, an involvement of this enzyme in immediate early events is unlikely. Therefore, we concentrated on a possible participation in the regulation of the cytokine-induced secondary response. As shown in FIG. 6, A, transfection of HUVEC with SPP2 siRNA, but not scrambled siRNA, suppresses the TNF-alpha-induced SPP2 mRNA levels by more than 85%, as determined by real-time PCR analysis. SiRNA-mediated silencing of SPP2 results in reduction of enzymatic activity: the TNF-alpha-triggered phosphatase activity is strongly suppressed in the presence of SPP2 siRNAs (FIG. 6, A). In addition, SPP2 siRNA, but not the control siRNA, completely abrogates the EGFP-SPP2 overexpression in HUVEC and does not alter an expression of EGFP-SPP1, when siRNAs are cotransfected together with the respective expression plasmid (FIG. 6, B) confirming the efficiency and specificity of SPP2 siRNAs. Inhibition of SPP2 expression by siRNA does not lead to a change in expression levels of other S1P-metabolizing enzymes, such as SPP1, SPHKs, or SPL (data not shown).

As shown in FIG. 7, C, the TNF-alpha-induced transcription of IL-1beta is significantly reduced by SPP2 siRNAs at all time points starting at 2.5 h, i.e., concurrent with the production of the SPP2 transcript. Thus, the first wave of IL-1beta mRNA up-regulation with a maximum around 4 h after stimulation is reduced by about 50%; the second wave of IL-1beta expression, starting at 16 h, is suppressed by more than 70%. Similar results are obtained with a second primer pair to IL-1beta (data not shown). To confirm the specificity of the effect of the SPP2 siRNAs, we used mismatch siRNAs as controls: they did not silence SPP2 expression and did not at all effect IL-1beta expression (data not shown). In line with the data on the mRNA level, expression of the TNF-alpha-induced 31/33-kDa IL-1beta precursor detected in HUVEC extracts at 16 h and 24 h post-stimulation is inhibited in the presence of SPP2 siRNAs (FIG. 7, D).

IL-8 is a second cytokine influenced by SPP2 silencing; TNF-alpha-induced transcription of IL-8 is found to be partially suppressed, however, only at late time points (16 h and 24 h) (FIG. 8, E). This partial inhibition at mRNA levels results in around 40% reduction of IL-8 protein levels in the supernatant (33.1±4.3 ng/ml and 18.1±2.2 ng/ml at 24 h time point for control siRNA and SPP2 siRNA, respectively). In contrast, SPP2 silencing does not affect significantly the TNF-alpha-triggered expression of an early responsive gene EGR-1, as well as TF, VCAM-1, MCP-1 (FIG. 8, E), RANTES, ICAM-1, TNF-alpha, and IL-6 (data not shown).

In order to determine whether the blocking effect of SPP2 siRNA on IL-1beta is specific for the TNF-alpha stimulus, HUVEC are exposed to LPS. Similar to the situation with TNF-alpha stimulation, LPS-driven expression of IL-1beta mRNA is reduced upon SPP2 silencing by around 60%, while up-regulation of MCP-1 and VCAM-1 is not affected (data not shown). In contrast to SPP2, neither SPHKs nor SPP1 silencing with validated siRNAs modulates the TNF-alpha-induced expression of IL-1beta or IL-8.

To explain why silencing of SPP2, but not of SPP1 influenced the inducible IL-1beta expression, we investigated whether (i) SPPs have differential subcellular localization in unstimulated HUVEC and/or in response to TNF-alpha; or (ii) able to modulate in different ways the TNF-alpha-triggered nuclear translocation of NFkappaB/RelA subunit. Transient expression of EGFP-tagged SPP1 or SPP2 in unstimulated cells, however, does not reveal any differences in their subcellular localization. The observed fine membranous reticular patterns are consistent with localization described in other cell types and similar to the staining of endoplasmic reticulum (ER) in live cells. Treatment of cells with TNF-alpha within 1.5 h does not induce subcellular re-localization of either EGFP-SPP1 or EGFP-SPP2 (microscopic monitoring with 10 min intervals; data not shown). Furthermore, overexpression of EGFP-SPP1 or EGFP-SPP2 does not alter the cytoplasmic localization of endogenous NFkappaB/RelA in unstimulated HUVEC and does not affect its nuclear translocation after TNF-alpha stimulation. Thus, the data indicate that forced overexpression of either SPP1 or SPP2 does not interfere with initial steps of NFkappaB activation.

7. Expression Signature of SPP2 in Lesional Skin of Patients with Psoriasis

Given the involvement of SPP2 in the regulation of IL-1beta, we asked whether this enzyme might be up-regulated in an inflammatory disease in man. Therefore, lesional and uninvolved nonlesional (NL) skin samples of patients with psoriasis—a chronic multifactorial inflammatory disease have been analyzed. SPP2 mRNA expression is significantly increased in the lesional skin of all patients compared to the corresponding NL skin. No significant modulations in the expression levels of SPP1, SPL, total SPHK1 or SPHK2 (FIG. 9) and their splice variants are detected (data not shown). Increased expression of IL-1beta mRNA is detected in psoriatic plaques (FIG. 9).

In sum, SPP2 mRNA expression is specifically triggered by inflammatory stimuli in a variety of cells (neutrophils, endothelial and epithelial cells, DC), but fails to be induced by growth-related factors, revealing a possible unique role of SPP2 during inflammation. In support of these findings, we demonstrate the existence of NFkappaB binding sites within the SPP2 promoter. Moreover, it is shown that silencing of the RelA subunit of NFkappaB by specific siRNAs results in strong reduction of the TNF-α-triggered SPP2 expression (as efficient as silencing by SPP2 siRNA) demonstrating the functional relevance of the NFkappaB binding. Small residual SPP2 mRNA levels detected after NFkappaB silencing can be explained either by incomplete NFkappaB knockdown or by involvement of another, but clearly subsidiary, transcription factor in the regulation of SPP2 transcription.

It is further shown that SPP2 silencing does not potentiate the up-regulation of any of the TNF-alpha or LPS-triggered genes in endothelial cells, including those where up-regulation is known to depend on SPHK1/S1P (e.g., VCAM-1, IL-6, MCP-1). In contrast, up-regulation of IL-1beta message and precursor protein by SPP2 silencing was strongly reduced. Thus, against expectations, SPP2 silencing leads to inhibition, rather than potentiation of a pro-inflammatory response in endothelial cells.

In summary, our data suggest an unexpected role of SPP2 in inflammatory signalling. Further studies will need to delineate the mechanism of SPP2 interaction with IL-1β transcription and its potential involvement in inflammation in additional cell types and diseases.

The following abbreviations are used herein:

DC dendritic cells
HKG house keeping genes

### RPMI Roswell-Park Memorial Institute

S1P sphingosine-1-phosphate
SPL SiP lyase
siRNA small-interfering RNA
Sph sphingosine
SPHK sphingosine kinase
SPHK1 sphingosine kinase 1
SPHK2 sphingosine kinase 2
SPP sphingosine-1-phosphate phosphatase
SPP1 S1P-specific phosphohydrolase 1
SPP2 S1P-specific phosphohydrolase 2
TF mRNAs encoding tissue factor

In the following Examples all temperatures are in degree (°) Celsius.

### EXAMPLE 1

### Cell Culture and Materials

Human umbilical vein endothelial cells (HUVEC) are cultured at 37° C. and 5% CO₂ in endothelial cell basal medium EGM™-2 (Clonetics, Cambrex BioScience, Walkersville, Md. USA). Cells are used for experiments up to five passages. For kinetic experiments, cells are seeded overnight in 6-well plates to reach confluency the next day. To measure IL-8, culture supernatants are taken at the indicated time points and analyzed by ELISA (R&D Systems, MN, USA) according to the manufacturer instructions. Primary human neutrophils are isolated from whole blood according to the method as described C. Ward et al, J. Biol. Chem. 274 (1999) 4309-4318, using dextran sedimentation and discontinuous plasma-Percoll gradients; cell purity and viability are routinely >95%. For kinetic studies, freshly isolated neutrophils are suspended at 2×10⁶ cells in RPMI containing 10% FCS in 12-well plates. Dendritic cells are prepared from highly enriched human monocytes according to a method as described in M. D. Saemann et al, J Leukoc Biol. 71 (2002) 238-246.

To induce final maturation, LPS (100 ng/ml) is added for 24 h. Cell lines used in this study are from ATCC Cell Biology Collection. Total RNA is isolated using Trizol reagent according to the protocol of the manufacturer (Invitrogen, Paisley, UK). Recombinant human VEGE165 and EGF are obtained from PromoKine (Heidelberg, Germany). TNF-α is from Genzyme Inc. (Cambridge, Mass.) and LPS from Sigma-Aldrich (Vienna, Austria).

### EXAMPLE 2

### Real-Time PCR Analysis

Expression profiling is performed by real-time PCR on ABI PRISM 7900HT Sequence Detection System (Applied Biosystems) according to a method as described in F. Bornancin, D. Mechtcheriakova, S. Stora, C. Graf, A. Wlachos, P. Devay, N. Urtz, T. Baumruker, A. Billich, Biochim. Biophys. Acta 1687 (2005) 31-43. In most experiments the SYBR Green detection system is used. Primers for sphingolipid-modifying enzymes and EF1A are designed using “Primer Express 2.x” software (Applied Biosystems). When possible, primers span exon-intron boundaries, so they do not detect signals from co-amplified DNA. For some low copy genes, the TaqMan probes or Assays-on-Demand (Applied Biosystems) are used in parallel to ensure reproducibility of results. The set of housekeeping genes (HKC) includes ubiquitin C (UBC) and β2-microglobulin (β2M), which are taken from the public real-time PCR primer and probe database at http://www.wzw.tum.de/gene-quantification, and eukaryotic translation elongation factor 1α1 (EF1A). Sequences of primers and probes are listed in TABLE 1 and TABLE 2 below. Each PCR reaction is performed in duplicate in a 25-μl final volume. Based on melting curve analysis no primer-dimers are generated during the applied 40 real-time PCR amplification cycles. For relative quantification, data are analyzed by ΔΔCT method using MS Excel and formulae described in the User Bulletin #2 ABI Prism 7700 Sequence detection System (Applied Biosystems). Expression levels of target genes in cells are normalized to the average of HKG and shown relative to unstimulated cells (time point “0”). For localization and sequence of primers for SPHK splice variants (SPHK1ₛₕₒᵣₜ, SPHK+14, and SPHK1+86, SPHK2_{long}, and SPHK2ₛₕₒᵣₜ) see FIG. 1 and TABLE 1.

TABLE 1
Endogenous levels of S1P and sphingosine
in stimulated HUVEC and neutrophils

| | |
|---|---|
| S1P | Sphingosine |

| | |
|---|---|
| Cells/stimulation | pmol/mg of total protein |

| HUVEC | | 13.0 ± 0.7 | 447.4 ± 26.0 |
|---|---|---|---|
| unstimulated | | | |
| 0.25 | h TNF-α | 15.5 ± 0.5 | 382.2 ± 8.4 |
| | | (p < 0.05)* | (p < 0.05) |
| 2.5 | h TNF-α | 13.8 ± 0.2 | 499.4 ± 34.1 |
| 6 | h TNF-α | 13.1 ± 0.2 | 471.6 ± 20.7 |
| 8 | h TNF-α | 12.9 ± 0.4 | 462.0 ± 10.1 |
| 16 | h TNF-α | 12.8 ± 0.5 | 428.1 ± 9.6 |
| Neutrophils | | 8.5 ± 1.3 | 31.2 ± 0.3 |
| unstimulated | | | |
| 0.25 | h TNF-α | 9.6 ± 1.3 | 29.7 ± 0.2 |
| 2.5 | h TNF-α | 9.8 ± 1.5 | 28.9 ± 0.8 |
| 6 | h TNF-α | 10.7 ± 1.0 | 26.5 ± 0.3 |
| 8 | h TNF-α | 11.8 ± 1.0 | 26.2 ± 0.2 |
| | | (p < 0.05) | (p < 0.05) |

*p-values are given for the comparison to unstimulated cells

TABLE 2
Transcriptional regulation of SPP2 and SPHK1 in activated cells
Cells are stimulated by various agonists within a 6 h time period.
Up-regulation of SPHK1 and SPP2 mRNAs is shown as maximal fold
increase relative to unstimulated cells. DC; shown is fold increase
relative to immature DC. Data are means ± S.D. of at least
three independent experiments.

Fold up-regulation

| Cells/stimulus | SPHK1 | SPP2 |
|---|---|---|
| HUVEC/TNF-α | <2 | 411 ± 36 |
| HUVEC/VEGF | <2 | <2 |
| HUVEC/EGF | <2 | <2 |
| HUVEC/PMA | <2 | 352 ± 41 |
| HUVEC/LPS | <2 | 290 ± 52 |
| Neutrophils/LPS | 160 ± 40 | 350 ± 75 |
| Neutrophils/EGF | 38 ± 7 | <2 |
| HL60/PMA | 15 ± 2 | 180 ± 21 |
| A549/IL-1β | <2 | 15 ± 2 |
| mature DC/LPS | 65 ± 12 | 174 ± 32 |

### EXAMPLE 3

### SPHK Activity in Neutrophils and HUVEC—In Vitro Assay

After stimulation, cells are collected by centrifugation and resuspended in lysis buffer (50 mM Tris, 10% glycerol, 0.05% Triton X-100, 1 mM DTT, 1 mM EDTA, 2 mM sodium vanadate, 10 mM sodium fluoride, pH adjusted to pH 7.4 and complemented with protease inhibitor mix (Roche)). The suspension obtained is frozen in liquid nitrogen and carried through two freeze/thaw cycles. Following centrifugation (16,000×g, 10 min, 4° C.), the supernatants are tested in the SPHK assay. Selective conditions for both SPHK1 and SPHK2 are used according to a method as described in A. Billich, F. Bornancin, P. Devay, D. Mechtcheriakova, N. Urtz, T. Baumruker, J. Biol. Chem. 278 (2003) 47408-47415. Radiolabeled S1P is visualized and quantified using a Molecular Dynamics Storm Phosphorlmager (Sunnyvale, Calif.). Enzyme activity is given as pmol of S1P formed per minute per milligram of total protein. Protein concentrations in cell lysates are determined using Bradford reagent (BioRad) with bovine albumin as reference.

### EXAMPLE 4

### Phosphatase Activity in Neutrophils and HUVEC—In Vitro Assay

Lipid phosphatase activity is estimated by the degree of dephosphorylation of radiolabeled S1P. The assay procedure used is adapted from the one described for SPP2 in vitro assay in C. Ogawa et al, J. Biol. Chem. 278 (2002) 1268-1272. After stimulation, cells are collected by centrifugation and resuspended in lysis buffer (50 mM HEPES, pH 7.3, containing 150 mM NaCl, 20% glycerol, 1 mM EDTA, 1 mM DTT and protease inhibitor mix (Roche)), followed by seven freeze/thaw cycles. In the case of neutrophils, lysates (20 μg-40 μg of total protein) are mixed with [3-³H]-S1P (0.25 μM final concentration, 0.5 μCi; American Radiolabeled Chemicals, Saint Louis, Mo., USA), prepared in mixed micelles with 0.1% fatty acid-free bovine serum albumin (Sigma, A-6003) according to a method as described in C. Ogawa et al, J. Biol. Chem. 278 (2002) 1268-1272, in a total reaction volume of 100 μl, and incubated at 37° C. for 15 min. For HUVEC, 5 μg of total protein is used; cold S1P at 2 μM prepared in micelles is added to the reaction mix. Then, 400 μl of PBS are added to the reaction mixture and lipids are extracted by additions of 707 μl of CHCl₃/CH₃OH/HCl/5M NaCl (300:300:7:100 v/v) with mixing. After sonication for 15 min, phases are separated by centrifugation, and the organic phase is recovered, dried, and dissolved in CHCl₃/CH₃OH/(19:1, v/v). The labeled lipids are resolved by thin-layer chromatography (TLC) on SilicaGel 60 high performance TLC plates (Merck) with 1-butanol/acetic acid/water (3:1:1, v/v). TLC plates are exposed to Kodak BioMax MR Film for 3-5 days at −80°. Bands are quantified by AlphaImager 2200 (Alpha Innotech Corporation). S1P phosphatase activity is expressed as pmol of sphingosine formed per minute per milligram of total protein.

### EXAMPLE 5

### In Silico Search for Transcription Factor Binding Sites

The proximal promoters of SPP2 and SPHK1 genes are analyzed using the 5′ flanking sequences (1.3 kb upstream of the translational start codon) obtained from the Celera database (SPP2 gene ID hCG21171; SPHK1 gene ID hCG30901). Transcription factor binding sites are identified by searching for particular promoter elements using TRANSFAC's MATCH program, version 6.4, with cut-offs adjusted to minimize false positive matches, see e.g. O. Kel-Margoulis et al, In Silico Biol. 3 (2003) 145-171.

### EXAMPLE 6

### Electrophoretic Mobility Shift Assay

Nuclear extracts from HUVEC stimulated with TNF-α for 1 h are prepared using Nuclear Extract Kit from Active Motif (Rixensart, Belgium). The double-stranded synthetic oligonucleotides are end-labeled using [□-³²P]dCTP (3000 Ci/Mol) (Amersham Life Sciences, Chalfont, UK) and Klenow polymerase (Bethesda Research Laboratories, Gaithersburg, Md.), and subsequently purified by gel electrophoresis (10% polyacrylamide gels). The binding reaction with nuclear extracts containing 1 μg of protein is performed according to a method as described in D. Mechtcheriakova et al, FASEB J. 15 (2001) 230-242. For competition experiments, a 50-fold molar excess of unlabeled double-stranded oligonucleotide is added to the reaction mixture. For supershift experiments, 1 μg of polyclonal rabbit antibody (RelA, cRel, or EGR-1, all from Santa Cruz Biotechnology, Santa Cruz, Calif.) is preincubated with nuclear extract on ice for 30 min before the addition of the radioactive probe. Protein-DNA complexes are separated in a 4% native polyacrylamide gel at 4° C. in Tris-borate-EDTA buffer. The following oligonucleotides are used in these experiments: hSPP2 OL_{SPP2/-753} 5′-CAGAAGGGACTTTCCTTCC-3′; hSPP2 OL_{SPP2/-853} 5′-CTGGTGGGGTTTTCCCAGTG-3′; hlg kappa chain OL_{NFκB} (NFκB consensus) 5′-CAGAGGGGGATTTCCAAGAG-3′; hTF OL_{Sp1} (Sp1 site from Tissue Factor promoter used as unspecific competitor) 5′-GGAGGCCGGGCAGGGGTGTGGACTCG-3′.

### EXAMPLE 7

### Inhibition of RelA and SPP2 Expression by Small Interfering RNA (siRNA)

HUVEC are seeded overnight in 6-well plates to reach 40% confluency the next day. Transient transfection of HUVEC with siRNAs is carried out by using LipofectAMINE and PLUS reagent (Invitrogen). Cells are incubated with transfection mixtures containing 100 nM (or 150 nM when indicated additionally) of siRNA duplexes, 5 μl of PLUS reagent, and 4 μl of LipofectAMINE in a total volume of 1 ml of medium 199 per well for 1 h 45 min. Cells are washed with the complete medium and cultured for 24 h before TNF-α (100 U/ml) is added for stimulation. Silencing of target genes is confirmed by real-time PCR analysis. To silence SPP2, two siRNA duplexes directed to different target sequences are combined at 75 nM each. Sequences of SPP2-directed siRNAs, corresponding mismatch controls and scrambled siRNAs, as well as siRNAs to other enzymes are listed in Supplementary Table 3. Validated siRNA against RelA is from Qiagen. Silencing of RelA/NFκB subunit and of IL-1β is additionally confirmed by Western blot analysis of total cell extracts using anti-RelA polyclonal antibodies (Santa Cruz Biotechnology) or anti-IL-1 monoclonal antibody (R&D Systems, UK), respectively. Membranes are reprobed with anti-actin monoclonal antibody from Sigma-Aldrich to ensure equal loading of samples.

### EXAMPLE 8

### Determination of S1P and Sphingosine in Cells

Cells (1-2×10⁶/sample) are lysed by freeze/thawing in 20 mM Tris-HCl, pH 7.4, containing 20% glycerol, 1 mM dithiothreitol, 1 mM EDTA, 1 mM sodium metavanadate and 15 mM sodium fluoride. Samples are spiked with internal standard (C17-sphingosine and C17-S1P; Avanti Polar Lipids, Alabaster, Ala.) and extracted with CH₃OH/CHCL₃ 2:1 containing 0.5% formic acid. The extracts obtained are subjected to acetylation with acetanhydride in pyridine (40° C., 20 min) according to a method as described in. E. V. Berdyshev et al, Analytical Biochemistry 339 (2005) 129-136. Following evaporation of solvents, samples are dissolved in CH₃OH/0.2% formic acid and subjected to high performance liquid chromatography (HPLC 1100; Agilent, Palo Alto, Calif.). In short, an Eclipse XDB C₈ column (5μ, 4.6×150 mm; Agilent) is eluted with a gradient (eluent A: 5 mM ammonium formiate+0.5% formic acid in CH₃OH/H₂O (80/20); eluent B: 5 mM ammonium formiate+0.5% formic acid in CH₃OH/CH₃CN/H₂O (49/50/1); 70 to 100% B in 10 min) at a flow of 0.5 ml/min at 40° C. Negative and positive ion electrospray-ionization with tandem mass spectroscopy (LC-MS/MS) is used to detect acetylated SIP and sphingosine, respectively, using an API 4000 QTrap instrument (MDS Sciex, Concord, Canada). The optimal collision energy for derivatized S1P and C17-S1P is found to be −28 and −26 V, respectively; the multiple reaction monitoring (MRM) transitions monitored are m/z 504.2/462.0 and m/z 490.1/448, 1, respectively. The optimal collision energy for derivatized sphingosine and C17-sphingosine is found to be +33 and +13V, respectively; the multiple reaction monitoring (MRM) transitions monitored are m/z 426.2/366.0 and m/z 412.2/250.0, respectively.

### EXAMPLE 9

### Live Cell Fluorescence Microscopy of HUVEC Transfected with EGFP-Tagged SPP1 and 2

SPP1 ORF (RefSeq NM 030791) is amplified by PCR from a human placenta cDNA library using the Advantage-GC 2 DNA polymerase with the primers 5′-CACCATGTCGCTGAGGCAGCGCCTGG and 5′-TCAAGAGATACCAATAAAGAAAAATATGTAAGG. The PCR fragment obtained is subcloned in pENTR/SD/D-TOPO. SPP2 (Genebank™ AF542512) ORF is amplified by 5′-extension of the partial cDNA sequence contained in the I.M.A.G.E. clone Id.4802628 (Genebank BG696302). This is achieved using the two following primers: 5′-CACCATGGCCGAGCTGCTGCGGAGCCTGCAGGATTCCCAGCTCGTCGCCCGCTTCCAGCGCCGC (forward) and 5′-TCAGGGTAATCCCAGAAACCTGTGAAGCATCG (reverse) and the Advantage-GC 2 DNA polymerase. The major PCR product, corresponding to the size of the full SPP2 ORF is purified, and further amplified with PfuUltra using the 5′-CACCATGGCCGAGCTGCTGCGGAG primer and the above mentioned reverse primer. The amplification product obtained is finally subcloned in pENTR/SD/D-TOPO. All constructs are checked by full sequencing. For expression, constructs are transferred to pEGFP-c1 (Clontech).

For live cell microscopy, HUVEC are seeded on Lab-Tek glass chamber slides (Nunc, NY, USA) 20 h prior to transfection, performed as above with siRNAs. For plasmids, 0.5 μg DNA per 1 ml of transfection mixture is used; for combination of siRNAs and encoding plasmid, 0.5 μg DNA and 150 nM of siRNA per 1 ml of transfection mixture are used. 24 h after transfection, living cells are analyzed with an inverted microscope (Axiovert 200M, Zeiss) equipped with a high resolution microscopy camera (AxioCam MRc, Zeiss) as well as oil DIC objectives (Plan-Neofluar 40x/1.30 and Plan-Apochromat 63x/1.40).

For immunofluorescence, HUVEC are grown on Lab-Tek glass chamber slides (Nunc, NY, USA) and transfected with plasmids encoding EGFP-SPP1 and EGFP-SPP2 as described above. After stimulation for 1 h with TNF-alpha, cells are washed twice with PBS, fixed for 10 min at room temperature with 4% paraformaldehyde, and permeabilized for 5 min with 0.5% Triton X-100 in PBS. Primaryantibodies against RelA/NF□B (Santa Cruz Biotechnology, Santa Cruz, Calif.) are diluted in PBS, 0.5% bovine serum albumin and incubated with the cells for 1 h at room temperature. Cells were washed with PBS and incubated with Texas Red-labeled goat anti-rabbit IgG (Jackson Immunoresearch, PA) for 1 h at room temperature. Cell are analyzed on Axiovert 200M equipped with a high resolution microscopy camera (AxioCam MRc, Zeiss) using oil DIC objective Plan-Neofluar 40×11.30. RelA/NF□B and EGFP-SPPs are detected by sequential monitoring of fluorescence using filter sets for rhodamine (excitation/emission 546/590 nm) and FITC (excitation/emission 450-490/515-565 nm), respectively. For estimation of fluorescence intensity within nuclei, multiple fields for each replicate of each experimental condition are imaged and at least 150 cells are quantified using software AxioVision 4.3. The average intensity values are displayed as densitometric mean.

### EXAMPLE 10

### Skin Biopsies and Total RNA Isolation

Punch biopsy samples (3 mm) are taken from lesional and non-lesional skin of patients with psoriasis. This study is performed in accordance with the guidelines of the Declaration of Helsinki. After approval of the investigational protocol by the institute and the local ethics committee of Medical University of Vienna, six patients with psoriasis were enrolled in the study after giving their informed consent. The skin biopsy specimens are snap-frozen in liquid nitrogen, and stored at −80° C. The biopsies are homogenized in 1 ml of Trizol reagent using tissue homogenizer Polytron PTA10-S and total RNA is isolated according to the protocol of the manufacturer (Life Technologies, Paisley, UK) using Phase Lock Gel extraction tubes (Eppendorf).

### EXAMPLE 11

### Statistical Analysis

All experiments are performed in duplicate or triplicate. The results are presented as means ±S.D. values of triplicate determinations or as means ±S.E.M. of at least three independent experiments with cells from different donors, each performed in duplicate. The statistical significance of the data is assessed with the two-tailed unpaired Student's t test; *p<0.05, **p<0.01, ***p<0.001, n.s., not significant.

What is claimed is:

1. A kit for diagnosing a disorder which is mediated by high levels of SPP2 or by SPP2 activity in a sample of an individual comprising:
a) means for detecting the level of SPP2 in a sample;
b) instructions how to use said kit in dendritic cells;
c) a detection means; and
d) a solid phase.

2. A kit according to claim 1, wherein the disorder is mediated by high levels of SPP2.

3. A kit according to claim 1, wherein the disorder is an inflammatory immune disorder.

4. A kit according to claim 1, wherein the disorder is psoriasis.

5. A method for diagnosing a disorder which is mediated by high levels of SPP2 or by SPP2 activity, comprising the steps of:
a) providing a sample of an individual;
b) determining the level of SPP2 in said sample;
c) comparing the level of SPP2 as determined in step b) with a reference level; and
d) diagnosing a disorder or disease, if the SPP2 level in the sample is elevated compared with the reference level.

6. A method according to claim 5, wherein the disorder is mediated by high levels of SPP2.

7. A method of monitoring the therapeutic efficacy in the treatment of an individual with a substance which is expected to have an effect on reducing or curing a disorder which is mediated by elevated SPP2 levels or SPP2 activity, which method comprises the steps of determining the level of SPP2 in dendritic cells in a sample of said individual suffering from such disease and comparing the level determined with the level of SPP2 prior to administration of said substance.

8. A method according to claim 7, wherein the disorder is mediated by high levels of SPP2.

9. An assay for identifying an agent that mediates a disorder which is mediated by elevated SPP2 levels or SPP2 activity, comprising the steps of:
a) determining the level of SPP2 in cells of a sample of an individual, in the absence and in the presence of a candidate compound which may be expected to modulate the level of SPP2;
b) identifying a candidate compound which modulates the level of SPP2 as determined in step a) as an agent; and
c) using such agent as a pharmaceutical in the treatment of disorders mediated by elevated SPP2 levels or SPP2 activity.

10. An assay according to claim 9, wherein the disorder is mediated by high levels of SPP2.
